(19) United States
(12) Patent Application Publication
(10) Pub. No.: US 2003/0172939 A1
(43) Pub. Date: Sep. 18, 2003

(54) Devices and methods using vibration to relieve discomfort

(76) Inventors: Aviv Hirchenbain, Ganel Yehuda (IL); Amir Porat, Ganel Yehuda (IL)

Correspondence Address: DR. MARK FRIEDMAN LTD., C/O BILL POLKINGHORN, DISCOVERY DISPATCH, 9003 FLORIN WAY, UPPER MARLBORO, MD, 20772, US

(21) Appl. No.: 10/388,518

(22) Filed: Mar. 17, 2003

Related U.S. Application Data
(63) Continuation-in-part of application No. 09/731,972, filed on Dec. 8, 2000.

(51) Int. Cl. A61M 1/00

(52) U.S. Cl. 128/867

(57) ABSTRACT

A method for relieving discomfort caused by congestion within a body cavity adjacent to at least one region of hard tissue employs a vibration generator which generates mechanical vibrations at a subsonic frequency. The vibration generator is brought into non-invasive mechanical engagement with the hard tissue to as to transmit vibrations through the hard tissue to at least part of the body cavity. Also described are devices for implementing the method of the invention.

This is a Continuation-In-Part of U.S. patent application Ser. No. 09/731,972 filed Dec. 8, 2000.

### FIELD AND BACKGROUND OF THE INVENTION

The present invention relates to pain relief and, in particular, it concerns devices and methods using vibration to relieve discomfort.

Various common forms of pain or discomfort are attributable to congestion or blockage of various body cavities or ducts. Examples of particular relevance to the present invention include congestion within the inner ear or Eustachian tube, and congestion within the roots or other cavities of teeth.

The ear (FIG. 1) contains two cavities, an outer ear cavity 10 and a middle ear 12. An eardrum 14 separates the two cavities. If there is a difference in pressure between these two cavities there will be a stress on eardrum 14. This stress can cause pain, tissue damage or hearing loss.

Outer ear cavity 10 is in direct contact with an external atmosphere (not shown) while the only contact between middle ear 12 and the external atmosphere is through an Eustachian tube 16. Eustachian tube 16 is a narrow tube between middle ear 12 and a nasopharynx/oropharynx (not shown). Under normal conditions, Eustachian tube 16 opens in response to yawning or swallowing. This preserves pressure equilibrium between middle ear 12 and the nasopharynx. The nasopharynx is in pressure equilibrium with the external atmosphere. Therefore, under normal conditions, middle ear 12 and outer ear cavity 10 are both in pressure equilibrium with the external atmosphere. Therefore the two cavities are in mutual pressure equilibrium and there is no stress on eardrum 14.

The pressure in outer ear cavity 10 immediately equilibrates to pressure changes in the external atmosphere. On the other hand, pressure changes in middle ear 12 require slow venting of air through narrow Eustachian tube 16. Therefore, sudden pressure changes in the external atmosphere may cause temporary pressure differential between outer ear cavity 10 and middle ear 12. This pressure differential stresses eardrum 14 and may result in pain (acute Barotrauma).

Barotrauma is often experienced by airplane passengers during landing and take off. For example, when an airliner ascends, the external pressure (cabin pressure) and the pressure in outer ear cavity 10 fall rapidly by as much as 10-15%. On the other hand, the reduction of pressure in middle ear 12 occurs much more slowly. This results in temporary over-pressure in middle ear 12. Until the excess pressure in middle ear 12 is fully vented, eardrum 14 will be stressed outward causing pain.

Eustachian tube 16 may become blocked in children or in adults with respiratory infections. This makes the equilibrating process between middle ear 10 and the external atmosphere exceedingly slow. On a long air journey, an air passenger with blocked Eustachian tube 16 may experience extreme and continuing pain due to pressure differential between outer ear cavity 10 and middle ear 12. Furthermore, this pressure differential can lead to chronic pain, hearing loss and tissue damage.

Mobley et al. (U.S. Pats. Nos. 5,467,784; 5,755,234; 5,819,745) developed a device that is intended to help airline passengers limit pressure differential between middle ear 12 and outer ear cavity 10. The Mobley et al. device is a passive earplug that shields outer ear cavity 10 from sudden pressure changes in the external atmosphere. The Mobley et al. device must be inserted into outer ear cavity 10 before any pressure change in the external atmosphere and must remain in outer ear cavity 10 continuously until pressure in middle ear 12 equilibrates with the pressure in the external atmosphere. If the Mobley et al. device is not inserted before pressure changes in the external atmosphere (take off) or if the Mobley et al. device falls out or is removed early then the Mobley et al. device gives no further benefit. Therefore the Mobley et al. device will fail for travelers who, at the beginning of their trip, were not aware of blockages that may exist in their Eustachian tube 16. The Mobley et al. device will also fail for travelers who prematurely remove the device. Furthermore, the Mobley et al. device can not be adjusted and retains pressure for a preset period that may not be appropriate for all users. Thus, for a passenger with blocked Eustachian tube 16, the preset pressure retention period of the Mobley et al. device may be too short. When the pressure retention period of the Mobley et al. device is too short, then the Mobley et al. device will not prevent Barotrauma. On the other hand the preset pressure retention period of the Mobley et al. device may be too long for passengers with clear Eustachian tube 16. When the pressure retention time of the Mobley et al. device is too long, then pressure in middle ear 12 will change faster in response to changes in external atmospheric pressure than pressure in outer ear cavity 10 causing (inverse) Barotrauma.

Another disadvantage of the Mobley et al. device is that it prevents pressure oscillations from reaching outer ear cavity 10. This reduces the hearing of the user of the Mobley et al. device. Reduced hearing causes inconvenience to the user who wishes to listen to music or join a conversation. Reduced hearing can also be a safety hazard because the user fails to receive important information such as announcements over an airplane's intercom.

Proetz (Proetz, A. W. “Allergy in middle and internal ear.” Ann Otol. 40: 67, 1931) developed a treatment for chronic blockage of Eustachian tube 16 by actively producing pressure oscillations (short-term overpressure) in the nasopharynx. Pressure in the nasopharynx affects middle ear 12 through Eustachian tube 16. Recently, Arick et al. (U.S. Pat. No. 5,419,762), Stangerup (U.S. Pat. No. 5,431,636) and Donaldson et al. (U.S. Pat. No. 5,950,631) developed devices to treat chronic ear diseases by controlling pressure in the nasopharynx. Controlling pressure in the nasopharynx can not alleviate Barotrauma in victims of blocked Eustachian tube 16 because the pressure from the nasopharynx can not traverse blocked Eustachian tube 16 to reach middle ear 12 fast enough to prevent pain or damage.

Liquid in middle ear 12 is common cause of reduced hearing in children. Liquid is drawn into middle ear 12 from surrounding tissue when there is negative pressure in middle ear 12. Negative pressure in middle ear 12 occurs when a child with partially blocked Eustachian tube 16 clears his nose by sucking. The Proetz methodology is not well suited for treating negative pressure in middle ear 12 of small children because applying pressure oscillations to the nasopharynx requires highly coordinated motor activity on the part of the user (stretching the neck and swallowing or blowing while holding the breath).

Otitis media is a painful inflammation of the middle ear and ranks second only to the common cold as the most frequent illness among children in the United States. Acute otitis media is usually accompanied by fever, swelling, inflammation of the eardrum and considerable pain. Otitis media develops when bacteria or viruses, usually associated with colds or sore throats, make their way up the Eustachian tube, from the upper part of the throat behind the nose to the middle ear. When fluid accumulates in the middle ear the condition is known as otitis media with effusion or “glue ear.” This condition can lead to hearing loss and affect a child's learning and language skills.

Nearly 70 percent of U.S. children will develop otitis media by age 2. Otitis media is a frequent problem in children because the eustachian tube is shorter, wider, and more horizontal than in adults. Many children will outgrow their susceptibility to the infection by age 5. Over half of those who experience acute otitis media will have repeated episodes and the condition may become chronic. Otitis media is the most common cause of hearing loss in the U.S. and represents a significant disability interfering with childhood learning processes. Estrada B, Infect Med 1997; 14(3):239-44. Otitis media accounts for over 35 percent of all visits to pediatricians each year and represents more than $3.5 billion in U.S. health care costs annually.

Turning now to dental pain, one of the common causes of toothache is a build-up of pressure in the root canal due to an abscess or other medical condition. Significant symptomatic relief would be provided by draining puss and/or excess fluids from the internal volume of the tooth.

It would therefore be highly advantageous to provide devices and corresponding methods for non-invasive application of vibrations at subsonic frequencies to relieve congestion and encourage pressure normalization within body cavities such as the middle ear, the Eustachian tube and dental root canals, thereby reducing discomfort.

### SUMMARY OF THE INVENTION

The present invention is a device and method which uses vibration to relieve discomfort.

According to the teachings of the present invention there is provided, a method for relieving discomfort caused by congestion within a body cavity adjacent to at least one region of hard tissue comprising the steps of: (a) providing a vibration generator configured to generate mechanical vibrations at a subsonic frequency; and (b) bringing the vibration generator into non-invasive mechanical engagement with the hard tissue to as to transmit vibrations through the hard tissue to at least part of the body cavity.

According to a further feature of the present invention, the vibration generator includes an electric motor.

According to a further feature of the present invention, the vibration generator includes a piezoelectric actuator.

According to a further feature of the present invention, the vibration generator is configured to generate vibrations at a plurality of subsonic frequencies.

According to a further feature of the present invention, the vibration generator further includes a heating device deployed for transferring heat to the hard tissue.

According to a further feature of the present invention, the body cavity is at least one of a middle ear and a Eustachian tube.

According to a further feature of the present invention, the hard tissue is the mastoid bone.

According to a further feature of the present invention, the vibration generator is further brought into non-invasive mechanical engagement with the base of an ear.

According to a further feature of the present invention, the body cavity is a dental root canal.

According to a further feature of the present invention, the vibration generator is inserted into a flexible housing having upper and lower bite surfaces for gripping between opposing teeth.

According to a further feature of the present invention, the vibration generator is inserted into a flexible housing having two substantially perpendicular contact surfaces configured for contacting a biting surface and a side surface of a tooth.

There is also provided according to the teachings of the present invention, a device for relieving discomfort caused by congestion within at least one of a middle ear and a Eustachian tube, the device comprising: (a) a vibration generator configured to generate mechanical vibrations at a subsonic frequency; and (b) a vibrator head associated with the vibration generator so as to be vibrated by the vibration generator, the vibrator head including: (i) a first contact region having an arch for engaging the base of an ear; and (ii) a second contact region having a convex surface spaced and oriented relative to the first contact region such that, when the arch engages the base of an ear, the convex surface is located adjacent to the mastoid bone.

According to a further feature of the present invention, the arch is implemented as a substantially arcuate concave edge.

According to a further feature of the present invention, the substantially arcuate concave edge is formed along an intersection between two non-parallel surfaces.

According to a further feature of the present invention, the convex surface is implemented as a partially spherical surface projecting from one of the two non-parallel surfaces.

According to a further feature of the present invention, the vibrator head further includes a third contact region formed as a partially spherical surface projecting from a second of the two non-parallel surfaces.

According to a further feature of the present invention, the vibration generator includes a user-operable control for varying at least one of a frequency and an amplitude of the vibrations.

There is also provided according to the teachings of the present invention, a device for relieving discomfort caused by congestion within a dental root canal, the device comprising: (a) a vibration generator element configured to generate mechanical vibrations at a subsonic frequency; and (b) a vibrator housing configured to receive the vibration generator element, the vibrator housing being formed primarily from a flexible polymer material and shaped to be gripped between an opposing pair of teeth.

According to a further feature of the present invention, there is also provided a control unit including a power supply, the control unit being connected to the vibration generator element via flexible electric wires.

According to a further feature of the present invention, there is also provided a power supply electrically connected for providing power to the vibration generator element, the power supply being located within the housing.

According to a further feature of the present invention, the vibration generator includes an electric motor.

According to a further feature of the present invention, the vibration generator includes a piezoelectric actuator.

According to a further feature of the present invention, the vibration generator includes a user-operable control for varying at least one of a frequency and an amplitude of the vibrations.

According to a further feature of the present invention, the housing is formed with at least a first abutment surface for abutting a biting surface of a tooth and a second abutment surface for abutting a side surface of a tooth, the second abutment surface being substantially perpendicular to the first abutment surface.

### BRIEF DESCRIPTION OF THE DRAWINGS

The invention is herein described, by way of example only, with reference to the accompanying drawings, wherein:

FIG. 1 is a schematic representation of the anatomy of the human ear;

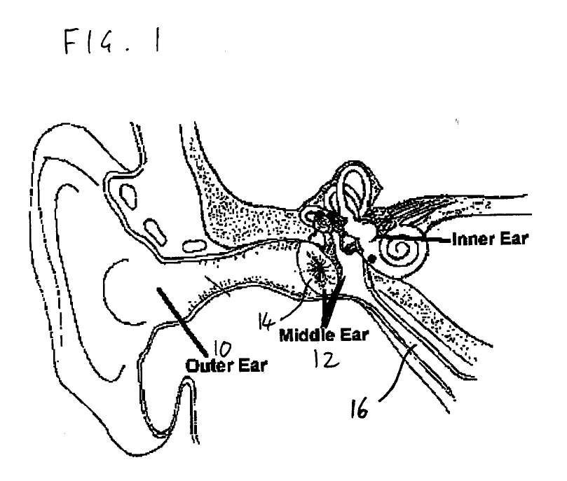

FIG. 2 is a schematic representation of the principle of operation of the present invention as applied to the ear;

FIGS. 3A-3E are front, side, rear, front isometric and rear isometric views, respectively, of a device, constructed and operative according to the teachings of the present invention, for applying vibrations to the ear and mastoid bone;

FIG. 4 is a schematic cross-sectional view of a first implementation of a device, constructed and operative according to the teachings of the present invention, for applying vibrations to a tooth;

FIG. 5 is a schematic cross-sectional view of a second implementation of a device, constructed and operative according to the teachings of the present invention, for applying vibrations to a tooth;

FIG. 6 is a schematic cross-sectional view of a third implementation of a device, constructed and operative according to the teachings of the present invention, for applying vibrations to a tooth; and

FIG. 7 is a schematic cross-sectional view of a fourth implementation of a device, constructed and operative according to the teachings of the present invention, for applying vibrations to a tooth.

### DESCRIPTION OF THE PREFERRED EMBODIMENTS

The present invention is a device and method which uses vibration to relieve discomfort.

The principles and operation of devices and methods according to the present invention may be better understood with reference to the drawings and the accompanying description.

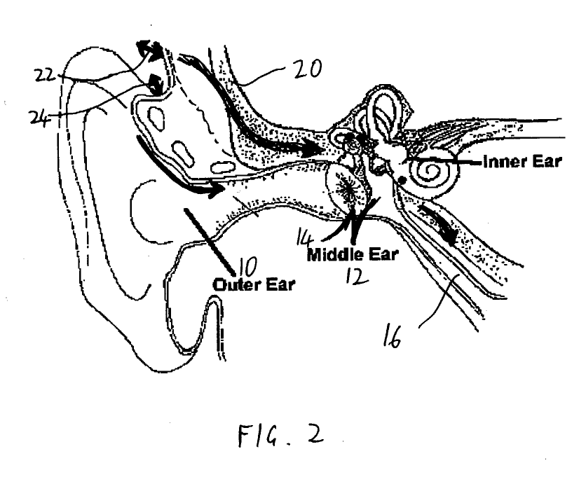

Referring now to the drawings, FIG. 2 shows the principle of operation of the present invention as applied to the ear. The method of the present invention is directed particularly towards relieving discomfort caused directly or indirectly by congestion within a body cavity adjacent to at least one region of hard tissue. In the case of the ear illustrated here, the congestion is typically congestion of one or both of middle ear 12 and Eustachian tube 16, such as caused by otitis media or other physiological or circumstantial conditions, as discussed above. In this case, the hard tissue used to transfer the vibrations is preferably the mastoid bone 20 which is accessible non-invasively by contact through a thin overlying layer of skin immediately behind the ear.

Generally speaking, the present invention employs a vibration generator, represented schematically in FIG. 2 by double-headed arrows 22, 24, configured to generate mechanical vibrations at at least one subsonic frequency. The vibration generator 22 is brought into non-invasive mechanical engagement with the hard tissue (in this case, at least mastoid bone 20) to as to transmit vibrations through the hard tissue (mastoid bone 20) to at least part of the body cavity (middle ear 12 and/or Eustachian tube 16). In the preferred case shown here, vibrations 24 are additionally applied to the base of the ear, thus being passed through the tissue of the outer ear wall itself towards ear drum 14.

The advantages of the present invention will be immediately apparent. The use of vibrations is non-damaging, non-invasive and has been found highly effective for clearing or relieving pressure in body cavities such as the inner ear and Eustachian tube, and even for freeing water caught in the outer ear after swimming. By employing externally accessible hard tissue to transmit the vibrations, vibrations can be used effectively to relieve congestion in regions of soft tissue not otherwise readily accessible. The result is a very high success rate in the immediate relief of symptoms due to pressure build-up, which may either replace or supplement other forms of treatment.

Before turning to features of the present invention in more detail, it will be useful to define certain terminology as used herein in the description and claims. Firstly, the invention is referred to as relieving congestion in a body cavity. The term “body cavity” is used herein in its broadest sense to refer to any volume within a living body, particularly a human body, within which a major proportion of the volume is normally occupied by gas or freely exchanged fluids. Body cavities thus defined include, but are not limited to, cavities within the structure of the ear, cavities of the nasal canals, sinuses, tooth root canals and other oral cavities, blood vessels and parts of the digestive tract. “Cavities” thus defined include both cavities which are part of normal physiology and those which result from pathological conditions (e.g., dental cavities resulting from tooth decay). Specifically, the present invention is believed of particular importance when applied to cases of barotrauma, otitis media and other pressure-related earaches. An additional particularly important field of application is symptomatic relief of toothaches, especially those related to abscesses.

In a further matter of terminology, reference is made herein to soft tissue and hard tissue. In this context, “hard tissue” is used to refer to body tissue which is sufficiently rigid to transfer vibrations effectively. Particular example are bone, cartilage, and teeth. “Soft tissue” is correspondingly defined as tissue which does not conform to the above definition of hard tissue.

Reference is also made to the vibration generator being in “non-invasive mechanical engagement” with the hard tissue. “Non-invasive” in this context means without making an incision into body tissue, but includes both external contact with the body and introduction into a body orifice. Most preferably, application of the vibrations is achieved through an external body surface, or at least a surface which is accessible when the mouth is open. “Mechanical engagement” in this context is used to refer to any mechanical interrelation which allows effective transfer of vibrations from the vibration generator to the hard tissue, but does not exclude the presence of intervening layers of soft tissue such as skin. Preferably, any intervening soft tissue has a total thickness of less than 5 mm in order to minimize interference with the transfer of vibrations.

Finally with regard to terminology, the vibrations used in the present invention are referred to as “subsonic”. This term, used interchangeably with “infrasonic”, is used to specify vibrations having a frequency below the audibility range of the human ear. Numerically, the frequency range for implementation of the present invention extends from 0.5 Hz up to 150 Hz. The preferred range of frequencies is between 15 and 70 Hz.

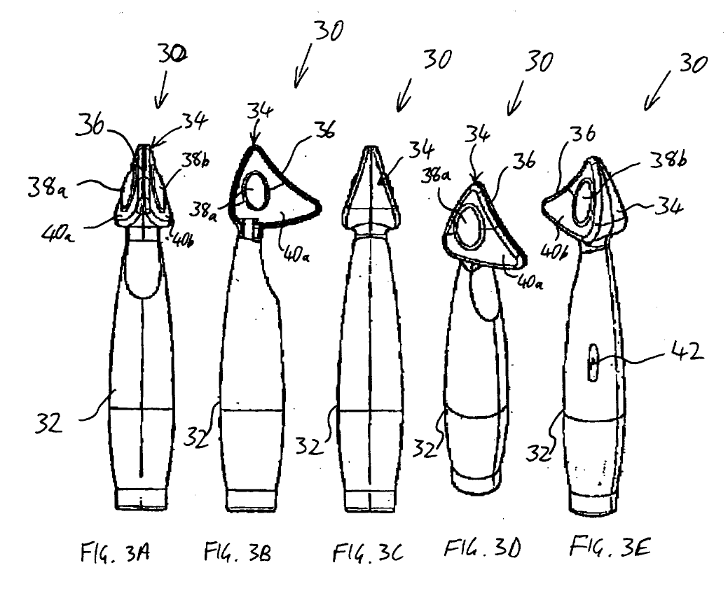

Turning now to FIGS. 3A-3E, there is shown a first preferred embodiment of the device of the present invention, generally designated 30. Device 30 includes a body 32 which houses a vibration generator configured to generate mechanical vibrations at a subsonic frequency. A vibrator head 34 is supported associated so as to be vibrated by the vibration generator. Vibrator head 34 includes a first contact region 36 having an arch for engaging the base of an ear. At least one, and preferably two, additional contact regions 38a, 38b, each having a convex surface, are spaced and oriented relative to first contact region 36 such that, when the arch engages the base of an ear, one of convex surfaces 38a, 38b is located adjacent to the mastoid bone.

The arch of first contact surface 36 is preferably implemented as a substantially arcuate concave edge. The radius of the arc is preferably between 3 and 30 centimeters, and most preferably between 5 and 15 centimeters. Preferably, the substantially arcuate concave edge is formed along an intersection between two non-parallel surfaces 40a and 40b. The convex surfaces 38a, 38b are implemented as a partially spherical surface projecting from one of the two non-parallel surfaces.

The vibration generator may be any conventional type of vibration generator, including but not limited to, and electric motor with an eccentrically weighted axis, and a piezoelectric actuator with a suitable electronic controller. Depending upon the type and size of the vibration generating element, part or all of the vibration generator may optionally be mounted within head 34 rather than in the handle. In the implementation shown here, an electric motor based implementation is believed to be particularly cost effective and efficient. The vibration generator preferably includes a user-operable control 42 (FIG. 3E) for varying at least one of a frequency and an amplitude of the vibrations. Appropriate arrangements for controlling the frequency and/or amplitude for a particular type of vibration generator will be clear to one ordinarily skilled in the art.

It has been observed that various different circumstances may affect the optimum choice of frequency for efficient relief of congestion within the body cavity. In order to increase the likelihood of supplying vibrations at or near the optimal frequency, the vibration generator may be configured to generate vibrations at a plurality of subsonic frequencies, as an addition or alternative to the user-operable control 42. Where an electronic controller is used, the controller may be configured to automatically vary the operating frequency between plural distinct frequencies or over a continuous range during use. Alternatively, a mechanical arrangement may be provided for varying the effective frequency of oscillations. One example of a mechanical arrangement is a secondary oscillator (e.g., a weight on a spring, not shown) mounted within head 30. The secondary oscillator is chosen to have a resonant frequency different from that produced by the vibration generator. The interaction between the vibration generator and the secondary oscillator then generates a complex motion which includes multiple frequency components. Various other mechanical frequency converters or frequency spreaders may also be used, as will be clear to one ordinarily skilled in the art.

Optionally, the effects of the vibrations of the present invention may be supplemented with one or more supplementary technique for facilitating relief of congestion within the body cavity. By way of one non-limiting example, the device may be combined with a heating device (not shown), for example located within head 34, deployed for transferring heat to the adjacent tissue.

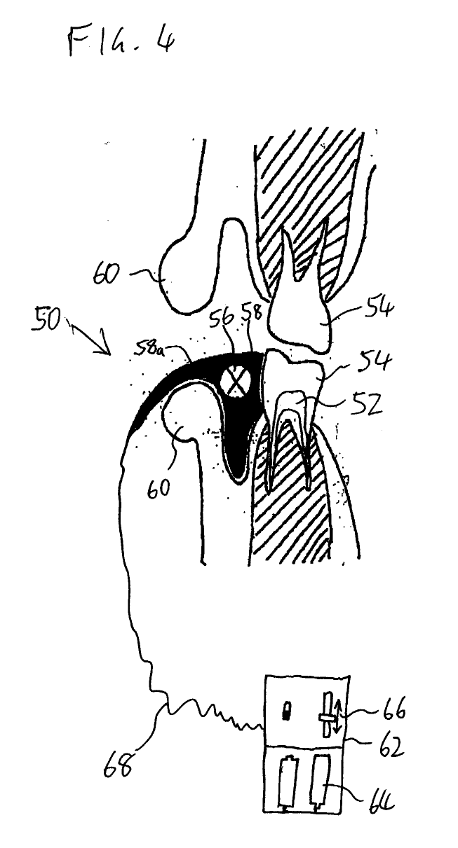

As mentioned earlier, the present invention is not limited to application to the ear. By way of a further particularly preferred example, an application of the invention for relief of toothache will now be described with reference to FIGS. 4-7.

Turning now to FIG. 4, this shows a device, generally designated 50, constructed and operative according to the teachings of the present invention, for relieving discomfort caused by congestion within a dental root canal 52 or other dental cavity.

In this case, in order to facilitate application of vibrations to a tooth 54 without requiring the user to hold a device in place manually, a vibration generating element 56 is preferably received directly within a vibrator housing 58. Vibrator housing 58 is formed primarily from a flexible polymer material and is shaped to fit against one or more surface of one or more teeth 54.

In the first preferred case shown here, housing 58 is configured to mount between tooth 54 and the adjacent lip 60, and has an outwardly extending flap 58a which allows the device to be held in place by closing opposing lips 60 together.

As in the previous embodiments, the vibration generating element 56 may be any suitable vibration generating element. In this implementation, a small cylindrical motor-based vibrator is particularly suitable.

In the implementation shown here, other elements required for operation of the vibration generating element are housed in a control unit 62. As in other implementations, control unit 62 typically includes a power supply 64, a user-operable frequency and/or amplitude control 66 and any other components (electronics etc.) which are necessary to operate the chosen type of vibration generating element in the desired manner. In this case, the external control unit 62 is connected to vibration generating element 56 via flexible electric wires 68.

Housing 58 may be made of any suitable material which is comfortable to position in the mouth and which is compatible with dental applications. Most preferably, housing 58 is a disposable molded piece of polymer of a type used in dental application. Suitable examples include, but are not limited to, soft vinyl Polysiloxane such as the product mix commercially available from Zhermack SPA (Italy) under product reference no. C201070.

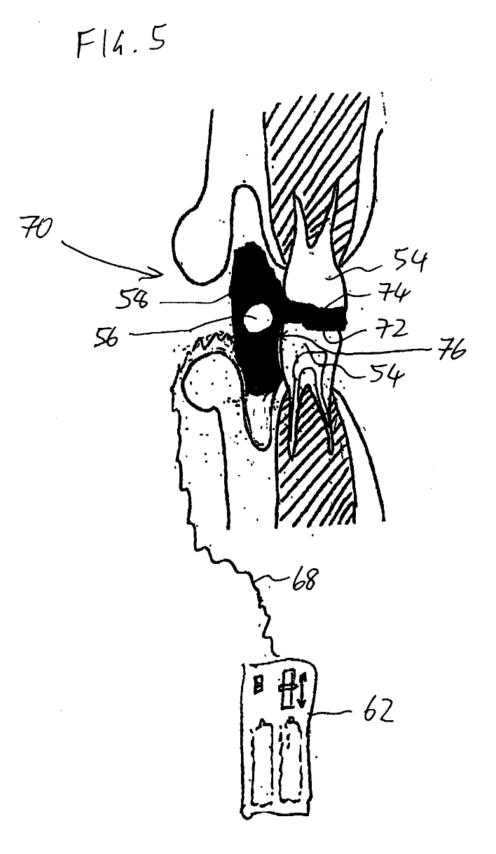

Turning now to FIG. 5, there is shown a second preferred implementation of a device 70, constructed and operative according to the teachings of the present invention, for dental applications. Device 70 is similar to device 60 and equivalent features are labeled similarly. Device 70 differs primarily in that housing 58 is here formed with substantially parallel upper and lower bite surfaces 72, 74 to facilitate gripping of housing 58 between an opposing pair of teeth 54. Additionally, housing 58 here features at least one additional abutment surface 76 deployed substantially perpendicular to abutment surface 72, so that abutment surfaces 72 and 76 contact a biting surface and a side surface of tooth 54, respectively. This arrangement is advantageous, particularly with a rotary vibration generating element 56, in that it efficiently imparts to the tooth vibrations in two orthogonal directions.

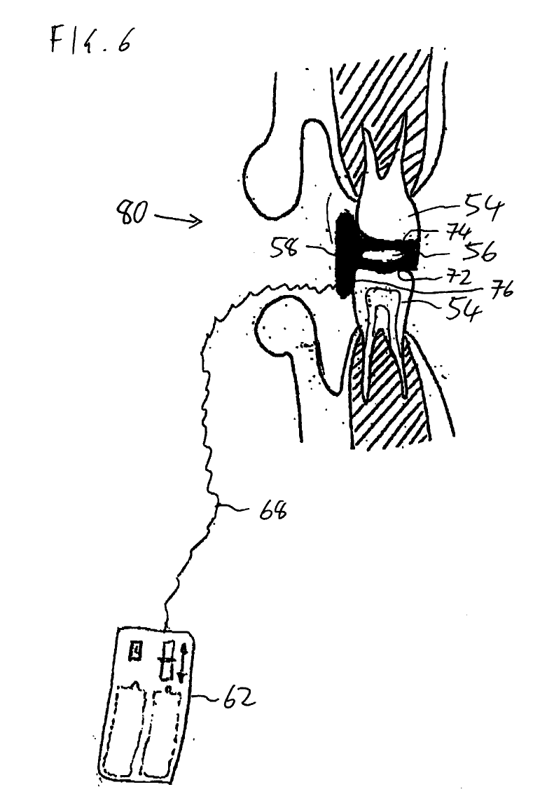

Turning now to FIG. 6, this shows a third preferred implementation of a device 80, constructed and operative according to the teachings of the present invention, for dental applications. Device 80 is similar to device 70 and equivalent features are labeled similarly. Device 80 differs primarily in that the vibration generating element 56 is here implemented as a substantially flat piezoelectric actuator. This allows positioning of the vibration generating element directly between bite surfaces 72 and 74 for grasping directly between the teeth.

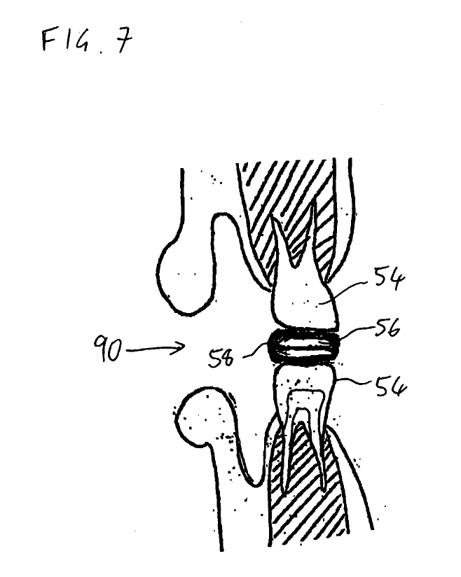

Turning finally to FIG. 7, this shows a fourth preferred implementation of a device 90, constructed and operative according to the teachings of the present invention, for dental applications. Device 90 is similar to device 80 and equivalent features are labeled similarly. Device 90 differs primarily in that the power supply 64 and any other required elements of the control system are located within housing 56 to form a self-contained unit. This implementation preferably employs flat-battery technology commercially available, for example, from PowerPaper Ltd. (Israel) and the entire device is preferably implemented as a single-use disposable unit.

It will be appreciated that the above descriptions are intended only to serve as examples, and that many other embodiments are possible within the spirit and the scope of the present invention.

What is claimed is:

1. A method for relieving discomfort caused by congestion within a body cavity adjacent to at least one region of hard tissue comprising the steps of:
(a) providing a vibration generator configured to generate mechanical vibrations at a subsonic frequency; and
(b) bringing said vibration generator into non-invasive mechanical engagement with the hard tissue to as to transmit vibrations through the hard tissue to at least part of the body cavity.

2. The method of claim 1, wherein said vibration generator includes an electric motor.

3. The method of claim 1, wherein said vibration generator includes a piezoelectric actuator.

4. The method of claim 1, wherein said vibration generator is configured to generate vibrations at a plurality of subsonic frequencies.

5. The method of claim 1, wherein said vibration generator further includes a heating device deployed for transferring heat to the hard tissue.

6. The method of claim 1, wherein the body cavity is at least one of a middle ear and a Eustachian tube.

7. The method of claim 4, wherein the hard tissue is the mastoid bone.

8. The method of claim 7, wherein said vibration generator is further brought into non-invasive mechanical engagement with the base of an ear.

9. The method of claim 1, wherein the body cavity is a dental root canal.

10. The method of claim 9, wherein said vibration generator is inserted into a flexible housing having upper and lower bite surfaces for gripping between opposing teeth.

11. The method of claim 9, wherein said vibration generator is inserted into a flexible housing having two substantially perpendicular contact surfaces configured for contacting a biting surface and a side surface of a tooth.

12. A device for relieving discomfort caused by congestion within at least one of a middle ear and a Eustachian tube, the device comprising:
(a) a vibration generator configured to generate mechanical vibrations at a subsonic frequency; and
(b) a vibrator head associated with said vibration generator so as to be vibrated by said vibration generator, said vibrator head including:
(i) a first contact region having an arch for engaging the base of an ear; and
(ii) a second contact region having a convex surface spaced and oriented relative to said first contact region such that, when said arch engages the base of an ear, said convex surface is located adjacent to the mastoid bone.

13. The device of claim 12, wherein said arch is implemented as a substantially arcuate concave edge.

14. The device of claim 13, wherein said substantially arcuate concave edge is formed along an intersection between two non-parallel surfaces.

15. The device of claim 14, wherein said convex surface is implemented as a partially spherical surface projecting from one of said two non-parallel surfaces.

16. The device of claim 15, wherein said vibrator head further includes a third contact region formed as a partially spherical surface projecting from a second of said two non-parallel surfaces.

17. The device of claim 12, wherein said vibration generator includes a user-operable control for varying at least one of a frequency and an amplitude of the vibrations.

18. A device for relieving discomfort caused by congestion within a dental root canal, the device comprising:
(a) a vibration generator element configured to generate mechanical vibrations at a subsonic frequency; and
(b) a vibrator housing configured to receive said vibration generator element, said vibrator housing being formed primarily from a flexible polymer material and shaped to be gripped between an opposing pair of teeth.

19. The device of claim 18, further comprising a control unit including a power supply, said control unit being connected to said vibration generator element via flexible electric wires.

20. The device of claim 18, further comprising a power supply electrically connected for providing power to said vibration generator element, said power supply being located within said housing.

21. The device of claim 18, wherein said vibration generator includes an electric motor.

22. The device of claim 18, wherein said vibration generator includes a piezoelectric actuator.

23. The device of claim 18, wherein said vibration generator includes a user-operable control for varying at least one of a frequency and an amplitude of the vibrations.

24. The device of claim 18, wherein said housing is formed with at least a first abutment surface for abutting a biting surface of a tooth and a second abutment surface for abutting a side surface of a tooth, said second abutment surface being substantially perpendicular to said first abutment surface.
